(19) United States
(12) Patent Application Publication
(10) Pub. No.: US 2017/0248582 A1
(43) Pub. Date: Aug. 31, 2017

(54) Mixed Spheroids of Melanocytes and Keratinocytes

(71) Applicants: Syntivia, Toulouse (FR); Centre Hospitalier Universitaire de Toulouse, Toulouse (FR); Universite Paul Sabatier - Toulouse III, Toulouse (FR); Centre National de la Recherche Scientifique, Paris (FR)

(72) Inventors: Marine Norlund, Toulouse (FR); Bernard Ducommun, Belberaud (FR); Philippe Bedos, Donneville (FR); Valérie Lobjois, Pujaudran (FR)

(73) Assignees: Syntivia, Toulouse (FR); Centre Hospitalier Universitaire de Toulouse, Toulouse (FR); Universite Paul Sabatier - Toulouse III, Toulouse (FR); Centre National de la Recherche Scientifique, Paris (FR)

(21) Appl. No.: 15/521,630

(22) PCT Filed: Oct. 29, 2015
(86) PCT No.: PCT/IB2015/058350

§ 371 Date: Apr. 25, 2017

(30) Foreign Application Priority Data
Oct. 30, 2014 (FR) 1460467

(51) Int. Cl. G01N 33/50; C12N 5/071

(52) U.S. Cl. CPC G01N 33/5088; C12N 5/0698; C12N 5/0626; C12N 5/063; G01N 33/5023; G01N 2500/10

(57) ABSTRACT

The present invention concerns the development of a model to evaluate active substances targeting the epidermis. It more particularly relates to the preparation of mixed spheroids of melanocytes and keratinocytes reproducing cell interactions occurring in the epidermis, to the spheroids as such and to the uses thereof.

The present invention is directed towards the development of a model for evaluating active substances targeting the epidermis. More specifically, it relates to the preparation of mixed spheroids of melanocytes and keratinocytes (also called mixed spheroids in the remainder hereof) reproducing cell interactions occurring within the epidermis, to the spheroids as such and to the uses thereof.

The epidermis is the uppermost surface layer of the skin. It is mostly formed of keratinocytes which proliferate and differentiate until they give dead cells called corneocytes eliminated by desquamation. Melanocytes, the specialised cells responsible for the production of melanic pigment are located in the epidermis at the basal stratum. The melanin produced by the melanocytes accumulates in vesicles known as melanosomes which are transferred to the keratinocytes via their numerous dendritic extensions. Pigmenting of the skin is the result of this process known as melanogenesis, leading to the production of melanin.

The epidermis fulfils several functions particularly including pigmentation of the skin and a barrier function chiefly ensured by the stratum corneum. These functions can be modulated by active substances.

For example, depigmenting products are commercially available for cosmetic or dermatological purposes to treat hyper-pigmentary disorders of melasma or solar lentigo type. The most known product, hydroquinone, has numerous side effects and is presently prohibited in cosmetics. The evidencing of new compounds that are possible alternatives to hydroquinone requires the use of biological models allowing evaluation of their depigmenting action.

At the current time, co-culture models of keratinocytes and melanocytes are the most widely used in this field. They allow the monitoring of different aspects of epidermal biology but do not provide information on the three-dimensional microenvironment of the epidermis. Models of reconstructed skin also exist more reliably mimicking the behaviour of these two cell types within the epidermis. However, reconstructed skin models are very cumbersome to prepare and also too costly for routine use in molecule screening.

It therefore appears necessary to develop three-dimensional epidermis models that are rapidly prepared and easy to handle for the evaluation of novel active substances.

It is within this context that the Applicant has developed spheroids comprising a mixed cell population of keratinocytes and melanocytes.

The present invention first relates to a method for preparing mixed spheroids of keratinocytes and melanocytes, characterized in that it comprises:
(a) suspending keratinocytes with melanocytes in a culture medium supplemented with a preparation of extra-cellular matrix proteins, preferably this preparation is contained in a concentration of between 1 to 95% by volume/volume (percentage expressed in volume of preparation relative to the total volume of the culture medium), preferably between 10 and 75% by volume/volume, and with an amount of 10 mM or less, preferably between 0.1 and 1 mM, of a salt selected from among calcium, manganese or magnesium, preferably calcium;
(b) forming spheroids from the mixture obtained at step (a); and
(c) incubating the spheroids obtained at step (b).

The cells, keratinocytes and melanocytes, that can be used in the method of the invention may be healthy or diseased cells depending on the desired use of the spheroids; if healthy cells are used, these may be healthy primary keratinocytes of NHEK type (Normal Human Epidermal Keratinocyte) and healthy primary melanocytes of NHEM type (Normal Human Epidermal Melanocyte) derived from biopsies or a commercial source. The method can be performed with melanocytes of any phototype between 1 and 6; it is within the reach of persons skilled in the art to choose the most suitable phototype for envisaged experimentation.

The ratio of keratinocytes to melanocytes is not critical for the preparation of the spheroids according to the invention; however, it is preferred to use a ratio as close as possible to that of the epidermis i.e. between 1 melanocyte per 1 keratinocyte and 1 melanocyte per 40 keratinocytes; preferably this ratio is between 1 melanocyte per 2 keratinocytes and 1 melanocyte per 5 keratinocytes.

The total number of seeded cells for preparation of a spheroid is not essential and is adapted as a function of the origin of the seeded cells; for example, to obtain spheroids of optimised size from healthy primary cells, it is preferable to seed between 100 and 5000 cells, preferably between 2000 and 3000 cells, for one spheroid. The cells are preferably seeded in 96-well plates.

The preparation of extra-cellular matrix proteins is preferably a preparation of sarcoma-derived protein extract e.g. the product sold by Corning Life Sciences under the trade name Matrigel®.

Step (b) is preferably performed by centrifugation, e.g. at between 50 and 2200 g for between 30 seconds and 15 minutes, preferably 6 minutes.

Incubation step (c) is conducted under conventional conditions i.e. at a temperature of between 35 and 38° C. According to one embodiment, this step comprises incubation lasting a time of about 1 h in the culture medium containing the preparation of extracellular matrix proteins such as Matrigel®, after which the salt is added and incubation continued preferably for at least 10 h.

By way of indication, the incubation at step (c) may last between 1 and 10 days, preferably between 2 and 5 days, according to the desired degree of keratinocyte differentiation.

According to one variant of the invention, the incubation of the spheroids is extended beyond 3 days to observe onset of keratinocyte modification (elongation of the cells, flattening of the nuclei, accumulation of keratin) in the centre of the spheroids (FIGS. 2 to 4). With this characteristic, the model of is great interest since it reproduces the differentiation gradient of the keratinocytes. The mixed spheroid therefore mimics the skin <<inside out>> (the most differentiated cells lying at the core of the spheroid) with a structure similar to the different strata of the skin (FIG. 4).

For use of the spheroids it is possible for them to be washed e.g. in PBS to remove the preparation of extracellular matrix proteins; this washing is preferably performed 24 h to 168 h after incubation, the time after which they are washed depending on the desired maturity of the mixed spheroids.

It is also possible to envisage removing the salt contained in the culture medium of the mixed spheroids before they are used.

Therefore, according to one particular embodiment of the method of the invention it comprises an additional step (d) to wash the mixed spheroids, preferably with PBS.

One advantage of the method of the invention is that it allows rapid production of a large number of spheroids having standardised characteristics of size and cell population, allowing the conducting of screening assays in series and comparison of the results obtained.

Various attempts to prepare spheroids, presented in the experimental section, allowed evidencing of the essential characteristics of the method. For example, the absence of salt and/or of preparation of extracellular matrix proteins, also containing growth factors in particular, or the preparation of a simple spheroid only containing one of the two cell types to which a suspension of the other cell type is added, do not lead to formation of the spheroids.

The present invention also relates to mixed spheroids of keratinocytes and melanocytes obtained with the preparation method of the invention; therefore, the mixed spheroids of keratinocytes and melanocytes according to the invention may comprise a preparation of extracellular matrix proteins in a concentration of between 1 and 95% by volume/volume (percentage expressed by volume of preparation relative to the total volume of the culture medium), preferably between 10 and 75%, and an amount of 10 mM or less, preferably between 0.1 and 1 mM, of a salt selected from among calcium, magnesium and manganese, able to be prepared with the preceding method; advantageously, the spheroids have an angular diameter of between 100 μm and 1000 μm and a regular shape. Preferably, the spheroids comprise a number of cells of between 100 cells and 5000 cells, preferably between 2000 and 3000 cells.

The present invention further relates to mixed spheroids which have a central mass corresponding to central differentiation of the keratinocytes.

The mixed spheroids of the invention reproduce keratinocyte/keratinocyte, keratinocyte/melanocyte interactions with the presence of dendritic extensions and the production of melanin by the melanocytes. They can therefore be used as models to evaluate the action of active molecules, in particular dermatological or cosmetic active molecules.

Various tests were conducted with mixed spheroids of the invention: after treatment of the mixed spheroids with compounds known to induce melanin production (IBMX, 3-isobutyl-1-methylxanthine) and inhibit melanin production (PTU, propylthiouracil), the amount of melanin (assayed by spectrophotometry) in relation total proteins was evaluated (see Example 5 and FIG. 8). 50% induction was visualised reproducibly after treatment with IBMX thereby validating the use of IBMX as reference molecule on the spheroid model. Treatment with PTU inhibits melanin production.

This direct quantification was completed with quantitative PCR assay; this method allows analysis of a large number of genes and hence evidencing of the action mechanisms of the molecules tested on the mixed spheroids. After a treatment time of 24 h with IBMX, all the pigmentation genes examined (TYR, MITF, PMEL and TYRP1) had been induced at genetic level (see Example 6 and FIG. 9). The MITF factor participating in the regulation of pigmentation genes and directly involved in response to IBMX from a mechanistic viewpoint, was increased by a factor of 5. The genes of melanin synthesis TYR and TYRP1 were also induced.

These results confirm that it is possible to quantify the melanin produced in the mixed spheroids of the invention after reference treatments. The amount of melanin is directly quantifiable via melanin assay and modulation was obtained after reference pro- or de-pigmenting treatments. In addition, transcriptional analysis is also possible, thereby completing evaluation of active substances.

The mixed spheroids of the invention are therefore of marked interest as model for the evaluation of pro- or de-pigmenting molecules.

This is not the sole application however for which the mixed spheroids of the invention are useful, since quantitative PCR can be performed for purposes other than pigmentation such as inflammation, proliferation, apoptosis . . . .

In this respect, additional tests on the mixed spheroids of the invention were carried out (see Example 7) to quantify differentiation changes of their constituent keratinocytes; these were performed using a genome chip on 64 genes specific to the metabolism of keratinocytes among which 15 are also expressed by melanocytes. The expression profiles were compared after a culture time of 3 days and 7 days (see Table 1 of Example 7). Several genes involved in epidermal adhesion and cohesion were over-expressed (CTNNA1, CDLN1, OCLN, EVPL, EPPK1 and PXN). Genes involved in the differentiation and specifically in the formation of the cornified envelope were also overexpressed (AQP3, STPLC1 et TGM1). The superimposition of these two cell profiles provides confirmation of the observations made at morphological level. It is also important to note the increased expression of several pro-inflammatory cytokines (IL6, IL8 and TNFA).

Alternatively, or in addition, for use of the model mixed spheroids to evaluate the action of active molecules, it may prove useful but not essential when preparing these spheroids to mark one or more constituents thereof in order to be able to monitor their change over time or when the spheroid is placed in contact with molecules to be tested. Marking may be of several types: marking of living cells with a staining agent before the formation of the spheroid, or antibody marking on a spheroid section or dissociated spheroids after spheroid formation. The type of marking will determine the analysis to be carried out.

The present invention also relates to use of the mixed spheroids of the invention to evaluate active substances able to act at the epidermis.

As previously set forth, through the functionality of their melanocytes, the mixed spheroids are a model of choice to evaluate the pigmenting action, whether via inhibition or stimulation, of formulated or encapsulated molecules and/or molecule mixtures or of dermatological or cosmetic products; to carry out this evaluation melanogenesis must be monitored and analysed. This monitoring can be conducted along several different lines: assay of the amount of melanin produced, quantification of the transfer of produced melanin towards the keratinocytes; the monitoring of melanogenesis possibly being completed with quantification of related genes using quantitative PCR.

In particular, the assay of melanin can be performed by photometry following a protocol such as described in Example 3.

Evaluation of the possible action of a molecule on pigmentation may also, or alternatively, be performed by real-time analysis of the transfer of melanosomes towards keratinocytes. This can be obtained by using marking such as described in Example 4, followed by analysis of the spheroids by deep 3D imaging; this evaluation can also be carried out using other techniques such as flow cytometry.

The use of mixed spheroids of the invention comprising a central mass may also concern studies on keratinisation and associated diseases (e.g. psoriasis, atopic dermatitis, . . . ) and can be used to screen molecules modulating differentiation of keratinocytes by measuring the thickness of the central mass under given kinetics and/or by transcriptome analysis of the genes involved in keratinocyte differentiation.

More generally, the mixed spheroids of the invention can be used to study disorders or pathologies resulting from dysfunction of keratogenesis, melanogenesis and/or melanin transfer, and for the screening of molecules able to correct such dysfunctions with a view to use thereof for cosmetic, dermatological or cancerological applications.

The present invention therefore particularly pertains to a method for screening molecules able to act on melanin production and/or on the transfer of melanosomes and/or to modulate expression of the genes involved in melanogenesis, comprising:

(i) preparing optionally marked spheroids using the method of the invention; preferably the method of the invention comprises washing step (d) of the mixed spheroids;

(ii) placing the spheroids prepared at step (i) in contact with one or more molecules to be tested;

(iii) selecting the molecules which modulate the amount of melanin and/or the transfer of melanosomes and/or expression of the genes involved in melanogenesis;

or a to method for screening molecules able to act on the differentiation of keratinocytes, comprising:

(i) preparing spheroids according to the invention, for example having a central mass and/or being marked;

(ii) placing the spheroids prepared at step (i) in contact with one or more molecules to be tested;

(iii) selecting molecules which modulate differentiation of keratinocytes.

### FIGURES

FIG. 1: A. image illustrating a mixed keratinocyte-melanocyte spheroid under transmitted light, having a cell ratio of 1 NHEM per 4 NHEKs. The scale bar represents 100 μm. B. Graph showing changes in volume of the mixed spheroids over 10 days.

FIG. 2: Images showing a spheroid of the invention at D3.

A. Cytokeratin 5 marking to evidence keratinocytes.

B. NKIbeteb (Pme117) marking to evidence melanocytes.

C. DAPI marking of cell nuclei.

D. EdU marking of proliferating cells. Proliferating cells can be seen on the periphery of the mixed spheroid.

E. Overlaying of the different markings.

The scale bar represents 100 μm.

FIG. 3: Images showing a spheroid of the invention at D7.

A. Cytokeratin 5 marking to evidence the keratinocytes. A gradient of CK5 expression can be seen corresponding to a differentiation gradient. It can be noted that the morphology of the keratinocytes is modified as a function of the differentiation gradient with elongation of the cells (arrow).

B. DAPI marking of cell nuclei. Modification of the morphology of the nuclei can be seen with elongation (arrow) and finally loss of nuclei with the differentiation gradient.

C. EdU marking of the nuclei of proliferating cells. Proliferating cells can be seen on the periphery of the mixed spheroid.

D. Overlaying of the different markings.

The scale bar represents 100 μm.

FIG. 4: Keratinocyte differentiation.

A. Diagram of epidermis organisation.

B. Skin section with Fontana Masson staining.

C. Detail of a cross-section of mixed spheroids at D7 with cytokeratin 5 marking in grey and EdU marking (nuclei of proliferating cells) in white.

FIG. 5: images of spheroids of the invention prepared with various melanocyte/keratinocyte ratios (1:20, 1:5 and 1:2). The scale bar represents 100 μm.

FIGS. 6A to 6E: images showing objects obtained with various attempts to prepare spheroids such as described in Example 2.

FIG. 7: Visualisation of melanin transfer in the mixed spheroids.

A. Analysis of a section of mixed spheroids with cytokeratin 5 marking (dark grey) and NKIbeteb (Pme117) marking (light grey). In detail, on the right, doubly marked cells can be seen (arrow). The scale bar represents 100 μm.

B. Analysis of a whole spheroid by SPIM imaging and 3D reconstitution using IMARIS software, with CellMask marking of the keratinocytes (dark grey) and CFDA marking of the melanocytes (light grey). In detail, on the right, doubly marked cells can be seen (arrow).

C. Analysis of spheroids disrupted by flow cytometry with cytokeratin 5 (FL4) and CFDA marking of the melanocytes (FL1). A double marked population can be seen (population surrounded by a dotted line).

FIG. 8: histogram representing the melanin concentration on mixed spheroids after IBMX treatment (compared with the solvent control used and PTU).

FIG. 9: histogram representing gene expression of the cells of a mixed spheroid of the invention quantified as explained in Example 6. The expression of the MITF gene directly involved in IBMX treatment is increased. Expression of the genes TYR, TYRP1 and PMEL under the control of MITF is also increased after IBMX treatment.

### EXAMPLE 1—PREPARATION OF SPHEROIDS OF THE INVENTION

### Method

The cells are detached by trypsinization and counted to prepare a mixed suspension of melanocytes et keratinocytes with 60 000 cells/mL in medium supplemented with 10% Matrigel® and 1 mM calcium. This suspension is then seeded in a 96-well plate previously coated with polyHEMA and centrifuged 6 min at 190 g at 4° C. The plate is incubated 1 h at 37° C., 5% CO₂, to obtain Matrigel® congealing, and culture medium is added to each well to hydrate the Matrigel®.

### EXAMPLE 2—CHARACTERIZATION OF THE SPHEROIDS OF THE INVENTION

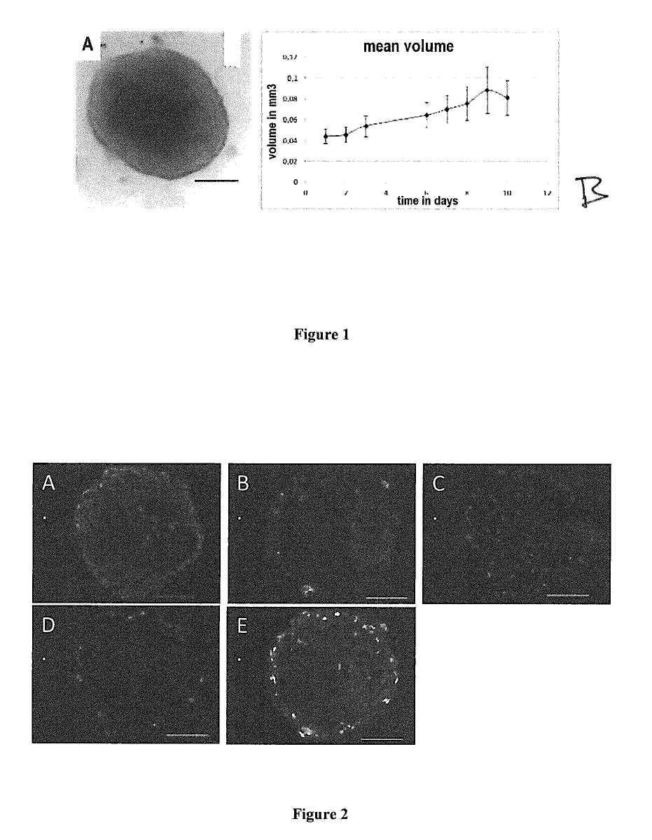

The spheroids thus produced have a diameter of about 250 μm after 3 days' culture and their volume doubles in 10 days (FIG. 1).

At D3, the marking method with NucView (caspase 3 marking) shows that no apoptosis occurs in the mixed spheroids. EdU markings allowed determination that the cells proliferate on the periphery of the spheroid. These cells are also marked with cytokeratin 5 and therefore correspond to keratinocytes (FIG. 2).

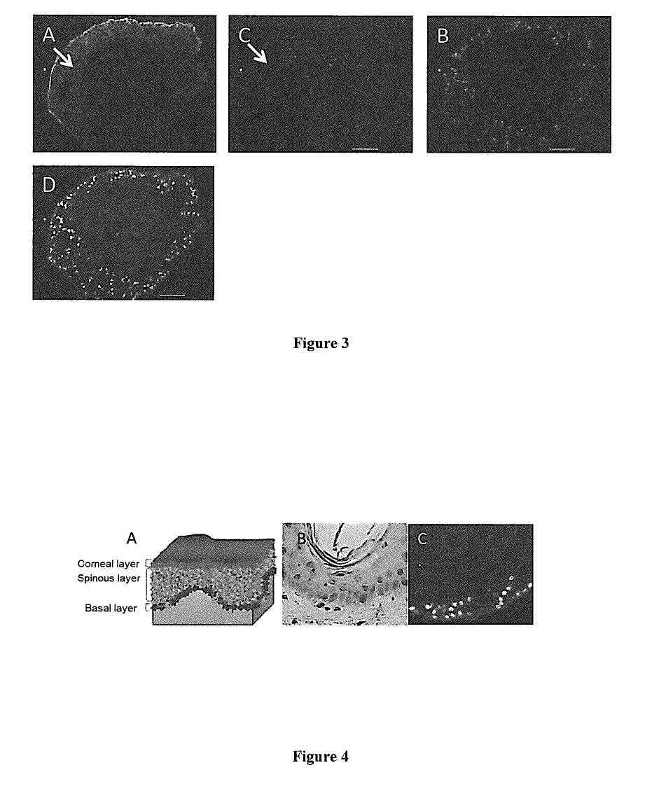

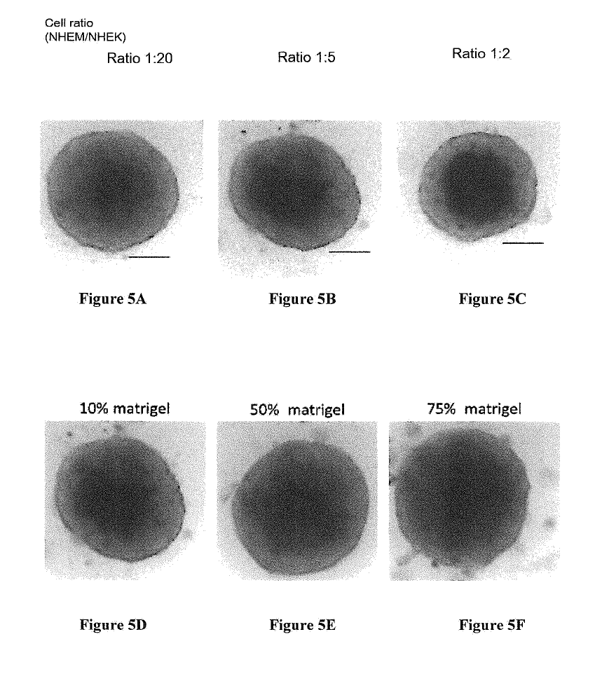

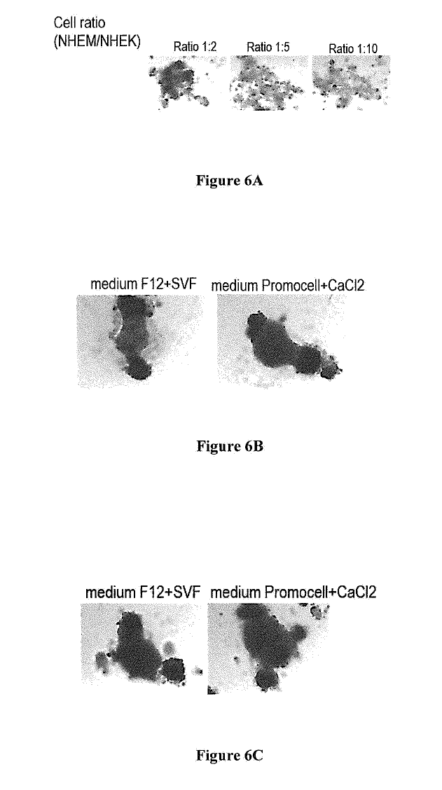

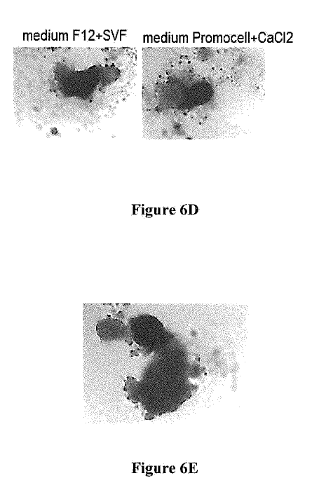

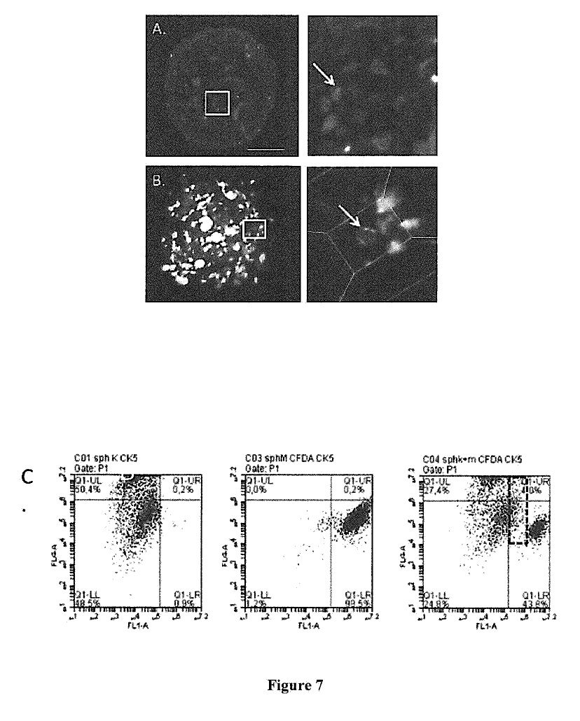

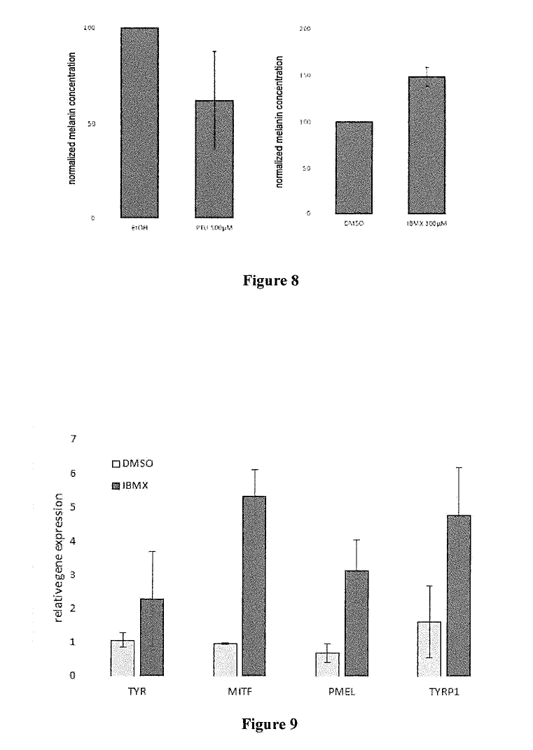

It is to be noted that, after several culture days, a central mass is seen in the spheroid and the morphology of the cells is modified (FIG. 3); this central mass is the result of differentiation of the keratinocytes (FIG. 4).

Spheroids of the invention were also prepared with various melanocyte/keratinocyte cell ratios (1:20, 1:5 and 1:2); they all exhibited satisfactory characteristics as illustrated in FIGS. 5A, 5B and 5C.

Other spheroids of the invention were prepared as in Example 1 using 50% and 75% Matrigel®; here again, the mixed spheroids obtained exhibited satisfactory characteristics; FIGS. 5D, 5E and 5F are photographs of these spheroids respectively prepared with 10%, 50% and 75% Matrigel®.

The following spheroid preparation conditions were also tested but did not allow spheroids to be obtained:
centrifugation method of a mixed NHEM-NHEK suspension in NHEK medium without Matrigel® and without calcium; with this method, the cells group together but do not form three-dimensional structures (FIG. 6A);
centrifugation method of mixed NHEM-NHEK suspension in NHEK medium supplemented with 1 mM CaCl₂ or in F12+SVF medium without Matrigel®: with this method, the cells form three-dimensional structures but remain segregated per cell type. The structure is not spherical and insufficiently reproducible for screening (FIG. 6B);
confrontation method of a NHEK-only spheroid with a suspension of NHEM in NHEK medium supplemented with 1 mM CaCl₂ or in F12+SVF medium without Matrigel®; the previously formed NHEK-only spheroids were prepared using the centrifugation method: with the confrontation method of a pre-existing spheroid with a cell suspension in a medium with calcium or in F12+SVF medium without Matrigel®, the cells form three-dimensional structures but remain segregated per cell type. The structure is not spherical and insufficiently reproducible for screening (FIG. 6C);
confrontation method of a NHEM-only spheroid with NHEK suspension in NHEK medium supplemented with 1 mM CaCl₂ or in F12+SVF medium without Matrigel®: the previously formed NHEM-only spheroids were prepared using the centrifugation method; with the confrontation method of a pre-existing spheroid with a cell suspension in a medium with calcium or in F12+SVF medium without Matrigel®, the cells form three-dimensional structures but remain segregated per cell type. The structure is not spherical and insufficiently reproducible for screening (FIG. 6D);
confrontation method of single spheroids (spheroids of NHEM and spheroids of MHEK respectively) in NHEK medium supplemented with 1 mM CaCl₂ without Matrigel®; the two previously formed single spheroids were prepared using the centrifugation method; with the confrontation method of two spheroids of each cell type in a medium with calcium but without Matrigel®, the cells form three-dimensional structures but remain segregated per cell type. The structure is not spherical and insufficiently reproducible for screening (FIG. 6E);
droplet method from a mixed NHEM-NHEK suspension in NHEK medium without calcium, supplemented or not with Matrigel® (10 to 50%). The conditions with Matrigel® did not form 3D structures; with this method the cells form three-dimensional structures but the structure is not spherical and insufficiently reproducible for screening;
centrifugation method of a mixed NHEM-NHEK suspension in NHEK medium without calcium and with a Matrigel® percentage varying from 10 to 50%: with this method, the cells form three-dimensional structures but remain segregated per cell type. The structure is not spherical and insufficiently reproducible for screening.

### EXAMPLE 3—ASSAY PROTOCOL OF MELANIN BY PHOTOMETRY

The cells were lysed in a solution of 1M NaOH+10% DMSO and incubated 30 min at 90° C. A standard range was prepared with synthetic melanin and read-out performed at a wavelength of 405 nm.

Assay of total proteins was performed on the lysate of melanin assay using the microBCA kit in accordance with the supplier's instructions. A standard range was prepared with BSA and read-out was performed at a wavelength of 605 nm.

The results are expressed in the form of a ratio between the assay of melanin and assay of total proteins.

### EXAMPLE 4—EXAMPLE OF A MARKING PROTOCOL FOR MIXED SPHEROIDS OF THE INVENTION

The mixed spheroids were prepared as explained in Example 1 with melanocytes previously marked with CFDA (carboxy-fluorescein diacetate, succinimidyl ester). The mixed spheroids thus formed were marked with CellMask. They were then washed, fixed in formalin, dehydrated and made transparent in a mixture of Benzyl-alcohol and benzyl-benzoate (BaBb). Analysis was performed using the light-sheet imaging technique.

### EXAMPLE 5—ASSAY OF MELANIN ON A MIXED SPHEROID AFTER TREATMENT WITH IBMX and PTU

24 h after seeding, the mixed spheroids of the invention such as prepared in Example 1, and after removal of Matrigel® by washing with PBS, were treated with 300 μM IBMX or 500 μM PTU for 5 days with renewal of treatment at 4 days. The melanin was assayed as explained in Example 3.

As illustrated in FIG. 8, the results obtained show that treatment with PTU leads to a decrease in the global amount of melanin in the mixed spheroid, in comparison with the solvent control (EtOH), and treatment with IBMX causes an increase in the total amount of melanin in the mixed spheroid as compared with the solvent control (DMSO).

### EXAMPLE 6—QUANTIFICATION OF GENE EXPRESSION BY QPCR

48 h after their seeding the mixed spheroids of the invention such as prepared in Example 1, and after removal of Matrigel® by washing with PBS, were treated with 300 μM IBMX for 24 h. The spheroids were collected and washed with PBS after 24 h treatment with IBMX. The RNAs were extracted and reverse-transcribed to cDNA. Quantitative PCR was then performed to quantify the expression of the genes MITF, TYR, TYRP1 and PMEL. The expression of these genes was normalised by expression of the GAPDH housekeeping gene and compared with the non-treated control.

The results obtained show that the MITF gene, directly involved in IBMX treatment, has its expression increased. The expression of the genes TYR, TYRP1 and PMEL, under the control of MITF, is also increased after treatment with IBMX (FIG. 9).

### EXAMPLE 7—EVALUATION OF THE GENE EXPRESSION PROFILE WITH GENOME CHIP

The gene expression profile of the spheroids was evaluated after 3 and 7 culture days on a genome chip following the protocol detailed below.

The mixed spheroids of the invention prepared as described in Example 1 were collected, washed in PBC at 3 days and 7 days respectively after seeding. The mRNAs were extracted followed by reverse transcription. Finally, the specific preparation steps for the chip (96 genes) applying the Fluidigm protocol were carried out. A pre-amplification step in the presence of all the primers used on the chip was performed. Each pre-amplified cDNA sample was then deposited with the mix in a 96-well plate following the plate layout allowing real-time PCR. In parallel, on another 96-well plate, each pair of primers was placed in a well following the plate layout. Each mix was then deposited either side of the chip. Mixing of the two mixes was performed by the IFC Controller and the chip then placed in the BioMark to conduct real-time PCR. The genome chip was used on 64 genes specific to the metabolism of keratinocytes of which 15 are also expressed by the melanocytes. Fluidigm software was used for data analysis. The results obtained are given in Table 1 below which shows that several genes involved in epidermal cohesion and differentiation are over-expressed after spheroid development between D3 and D7 as illustrated in FIG. 3; the inflammation genes are also over-expressed:

TABLE I
Table showing genes over-expressed between culture Days 3 and 7

| | % over- | |
|---|---|---|
| Gene | expression | Action |
| CTNNA1 | +50% | Alpha-catenin binds to Cadherin to promote cell |
| | | interactions |
| CDLN1 | +280% | Claudin is a membrane protein acting at tight |
| | | junctions |
| OCLN | +270% | Occludin is a membrane protein acting at tight |
| | | junctions |
| EVPL | +60% | Envoplakin is a protein involved in the |
| | | formation of desmosomes |
| EPPK1 | +540% | Epiplakin is a protein involved in the formation |
| | | of desmosomes. It also seems to be involved in |
| | | cell differentiation |
| PXN | +40% | Paxillin acts in protein complexes belonging to |
| | | the cytoskeleton. It is located on the cytoplasmic |
| | | surface of regions specialised in the attachment |
| | | of cells to the extracellular matrix |
| AQP3 | +90% | In man, the circulation of water within the skin |
| | | occurs via a specific Aquaporin called AQP3. It |
| | | plays an essential role in the formation of the |
| | | hydrolipid barrier |
| TGM1 | +110% | Transglutaminase is involved in the formation of |
| | | the cornified envelope creating cross-links |
| | | between structural proteins including involucrin |
| | | and thereby rigidifying the stratum corneum. |
| STPL Cl | +40% | Serine palmitoyltransferase is an enzyme acting |
| | | in the biosynthesis of sphingolipids which |
| | | concentrate in the stratum corneum. It is |
| | | therefore a marker of epidermal differentiation |
| Il8 | +390% | Pro-inflammatory cytokine |
| IL6 | +710% | Pro-inflammatory cytokine |
| TNFA | +710% | Pro-inflammatory cytokine |

1. Method for preparing mixed spheroids of keratinocytes and melanocytes, characterized in that it comprises:
(a) suspending keratinocytes with melanocytes in a culture medium supplemented with a preparation of extracellular matrix proteins at a concentration of between 1 and 95% by volume/volume and an amount of 10 mM or less of a salt selected from among calcium, manganese or magnesium;
(b) forming spheroids from the mixture obtained at step (a); and
(c) incubating the spheroids obtained at step (b).

2. The method according to claim 1, characterized in that said suspension at step (a) is prepared with 1 melanocyte per 1 keratinocyte to 1 melanocyte per 40 keratinocytes.

3. The method according to claim 1, characterized in that the keratinocytes and melanocytes are healthy primary cells and are seeded in a number of between 100 and 5000 cells per spheroid.

4. The method according to claim 1, characterized in that the preparation of extracellular matrix proteins is MATRIGEL.

5. The method according claim 1, characterized in that the salt is calcium.

6. The method according to claim 1, characterized in that the incubation at step (c) lasts between 1 and 10 days.

7. The method according to claim 1, characterized in that it further comprises a marking step.

8. The method according to claim 1, characterized in that it comprises a step (d) to wash the mixed spheroids.

9. A mixed spheroid of keratinocytes and melanocytes able to be obtained with the method according to claim 1.

10. The spheroid according to claim 9, characterized in that it has an angular diameter of between 100 and 1000 μm and is of regular shape.

11. The spheroid according to claim 9, characterized in that it has a central mass.

12. The spheroid according to claim 9, characterized in that at least one of the constituents thereof is marked.

13. Use of a spheroid according to claim 9 to evaluate the activity of compounds acting at the epidermis.

14. Use of a spheroid according to claim 9 to test the activity of compounds on melanin production and/or transfer of melanosomes and/or the expression of genes involved in melanogenesis.

15. Use of a spheroid according to claim 9 to test the activity of compounds on the differentiation of keratinocytes.

16. A method for screening molecules able to act on melanin production and/or on the transfer of melanosomes and/or on the expression of genes involved in melanogenesis, comprising:
(i) preparing spheroids according to claim 9;
(ii) placing the spheroids prepared at step (i) in contact with one or more molecules to be tested;
(iii) selecting molecules which modulate melanin production and/or the transfer of melanosomes and/or the expression of genes involved in melanogenesis.

17. A method for screening molecules able to act on the differentiation of keratinocytes, comprising:
(i) preparing spheroids according to claim 9;
(ii) placing the spheroids prepared at step (i) in contact with one or more molecules to be tested;
(iii) selecting molecules which modulate the differentiation of keratinocytes.
